(19) United States
(12) Patent Application Publication
(10) Pub. No.: US 2010/0204794 A1
(43) Pub. Date: Aug. 12, 2010

(54) PROSTHETIC VERTEBRAL BODY

(76) Inventors: Peter Jarzem, Town of Mount Royal (CA); Jean Ouellet, Montreal (CA); Marco Ferrone, Montreal (CA)

Correspondence Address: OGILVY RENAULT LLP, 1, Place Ville Marie, SUITE 2500, MONTREAL, QC, H3B 1R1, CA

(21) Appl. No.: 12/663,129

(22) PCT Filed: Jun. 6, 2008
(86) PCT No.: PCT/CA2008/001087

§ 371 Date: Apr. 23, 2010

Related U.S. Application Data
(60) Provisional application No. 60/942,334, filed on Jun. 6, 2007.

(51) Int. Cl. A61F 2/44

(52) U.S. Cl. 623/17.12

(57) ABSTRACT

A vertebral prosthesis (10) comprising opposed ends (14,16) interconnected by a tubular side wall (12), which enclose a cavity therewithin. An inlet port (18) in fluid flow communication with the cavity permits injection of a hardenable fluid into the cavity. The tubular side wall has an expanding bellows configuration which allows for expansion of the vertebral prosthesis in an axial direction (23) such that the ends are displaced away from each other when the cavity is filled with the hardenable fluid, thereby axially expanding the vertebral prosthesis from a collapsed position to an expanded position in order to fill a space between adjacent vertebral bodies (11,13). The vertebral prosthesis (10) has an expansion ratio, defined by a total axial height of the vertebral prosthesis in the expanded position divided by a total axial height of the vertebral prosthesis in the collapsed position, that is greater that 200 percent.

### CROSS-REFERENCE TO RELATED APPLICATIONS

The present patent application claims priority on U.S. provisional patent application No. 60/942,334 filed Jun. 6, 2007, the entire contents of which is incorporated herein by reference.

### TECHNICAL FIELD

The present invention relates generally to prosthetic vertebral bodies, and more particularly relates to an expandable prosthetic vertebral body.

### BACKGROUND OF THE INVENTION

Vertebrectomy, the excision of a vertebra, is often employed to address several conditions which severely weaken the spinal vertebrae, in order to decompress the spinal cord and/or to stabilize the vertebral column, and thereby reducing the likelihood that a weakened vertebra may fracture and cause significant nerve injury. These conditions can include, but are certainly not limited to, cancer, infection, bone disease and genetic bone malformation, for example. Trauma or fractures can also necessitate such an excision of a vertebra.

Most known operative techniques for the excision of a vertebra, or a part thereof, are limited by the relatively restricted access to the vertebra which is to be removed and subsequently replaced and/or reconstructed. Most commonly, vertebrae are removed either from an anterior approach (i.e. via the front of a patient) or a posterior approach (i.e. via the back of the patient). Anterior approach techniques provide the widest access to the vertebra or vertebrae to be excised, however are sometimes associated with comorbidities with respect to the thoracotomy. Posterior approach techniques are generally preferred and are more frequently used as they are typically less morbid, however they imply considerable constraints in terms of limited access, as the vertebra must be excised and replaced with a suitable prosthetic replacement without damaging the nerve roots.

Prosthetic vertebral body “cages” have been used to replace the damaged vertebra, once removed. However, in order to fill the space created by the excised vertebra, such cages must typically be sufficiently large. Thus, most known vertebral body replacement cages are intended to be placed using an anterior approach, which allows for greater access. Such known cages cannot easily be positioned without causing unwanted damage, given the tight space constraints. The installation of such known vertebral cages via the patient's back (i.e. using a posterior approach) often requires resection of a nerve root in order to create a space large enough to permit cage entry. Present cages therefore do not have sufficiently small size envelopes (whether diameter, length, etc.) or sufficient collapsibility, to readily permit entry thereof between nerve roots if installed using a posterior approach.

While some existing prosthetic vertebral cages can be expanded to fill a space left following excision of a vertebra, these are typically rigid, metallic structures which use a jack or a threaded shaft to expand. Another known vertebral prosthesis uses an expanding bellows-type joint between two end housings, however even with the expandable joint fully compressed, the overall size of the end housings when stacked together remains significant enough to prevent its insertion via a posterior approach. Further, this expanding vertebral prosthesis is relatively complex, and thus expensive, given additional stabilization provided by the addition of a rigid suspension plate surrounded by an elastomeric suspension medium, which is disposed within each of the rigid end housings. This additional stabilization provided by the suspension system employed results in a cage structure which is mobile relative to the vertebrae on either side thereof, which can be disadvantageous in certain applications.

Accordingly there remains a need for an improved prosthetic vertebral body which is sufficiently small upon insertion to permit it to be positioned in place via relatively small access ports or pathways, including via a posterior approach, while nevertheless being able to sufficiently expand to fill a much larger space left by an excised vertebra, or a portion thereof, and which is able adapt to various bone geometries upon expansion.

### SUMMARY OF THE INVENTION

It is an object of the present invention to provide an improved prosthetic vertebral body.

In accordance with one aspect of the present invention, there is provided a vertebral prosthesis comprising opposed first and second ends interconnected by a tubular side wall, the first and second ends and the tubular side wall enclose a cavity therewithin, at least one inlet port in fluid flow communication with said cavity permits injection of a hardenable fluid into said cavity, the first and second ends including outer surfaces thereon which are respectively adapted to abut adjacent vertebral bodies for engagement therewith, the tubular side wall having an expanding bellows configuration which allows expansion of the vertebral prosthesis in axial direction such that the first and second ends are displaced away from each other when the cavity is filled with the hardenable fluid, the vertebral prosthesis being thereby axially expandable from a collapsed position to an expanded position in order to fill a space between said adjacent vertebral bodies, the vertebral prosthesis having an expansion ratio defined by a total axial height of the vertebral prosthesis in the expanded position divided by a total axial height of the vertebral prosthesis in the collapsed position, the expansion ratio being greater that 200 percent.

There is also provided, in accordance with another aspect of the present invention, a vertebral prosthesis for replacement of at least one vertebral body excised from between two other vertebral bodies, the vertebral prosthesis comprising: opposed end plates including outer surfaces thereon which face in opposite directions and are respectively adapted to abut said two other vertebral bodies for fastening engagement therewith; a tubular side wall interconnecting the end plates to define an enclosed cavity therewithin, the tubular side wall having an expanding configuration allowing expansion of the vertebral prosthesis in a axial direction such that the end plates are displaced away from each other, when said cavity is filled with a hardenable fluid, such that the vertebral prosthesis expands from a collapsed position to an expanded position thereof in order to fill a space left by the at least one excised vertebral body; and wherein the vertebral prosthesis has an expansion ratio defined by a total axial height of the vertebral prosthesis in the expanded position divided by a total axial height of the vertebral prosthesis in the collapsed position, the expansion ratio being greater that 200 percent.

### BRIEF DESCRIPTION OF THE DRAWINGS

Further features and advantages of the present invention will become apparent from the following detailed description, taken in combination with the appended drawings, in which:

FIG. 1 is a perspective view of a prosthetic vertebral body in accordance with one aspect of the present invention, shown in an expanded position between two vertebrae;

FIG. 2A is a perspective view of the prosthetic vertebral body of FIG. 1, shown in a collapsed position;

FIG. 2B is a perspective view of the prosthetic vertebral body of FIG. 1, shown in an expanded position;

FIG. 3 is a perspective view of a prosthetic vertebral body in accordance with an alternate embodiment of the present invention, having a central first tab for pedicle screw capture and a second tab mounted to the end plate;

FIG. 4A is a perspective view of the prosthetic vertebral body of FIG. 1;

FIG. 4B is an enlarged detailed view of portion 4B of the side wall of the prosthetic vertebral body of FIG. 4A;

FIG. 5 is a perspective view of a installation system used with the prosthetic vertebral body of the present invention;

FIGS. 6A to 6D are schematic cross-sectional profiles of embodiments of the prosthetic vertebral body of the present invention; and

FIG. 7 is a side perspective view of a prosthetic vertebral body in accordance with another aspect of the present invention, engaged to an insertion handle.

### DETAILED DESCRIPTION OF A PREFERRED EMBODIMENT

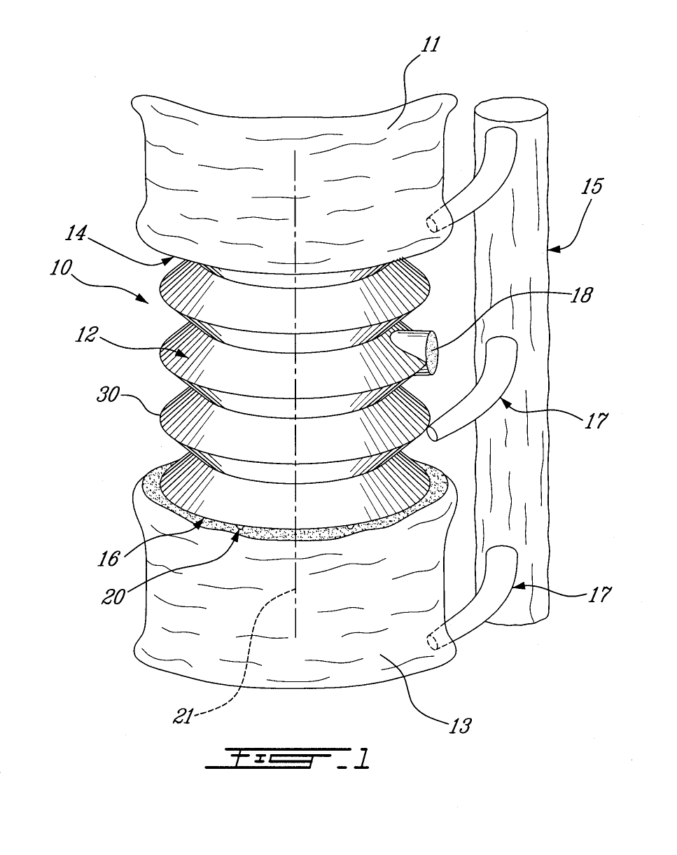

Referring to FIG. 1, a vertebral prosthesis 10 in accordance with one embodiment of the present invention is shown in an expanded position, installed in place between two vertebrae 11 and 13. The spinal cord 15 is shown schematically and includes nerve roots 17 for each vertebrae. The vertebral prosthesis 10 is thus used to replace one or more excised vertebra, a portion of a vertebra and/or to stabilize and fix the spinal column of a patient, especially in conjunction with a spinal resection in which the vertebral prosthesis or implant 10 is braced between upper and lower vertebrae, such as vertebrae 11 and 13 shown in FIG. 1. Vertebral implants are sometimes referred to as “cages”, because they have traditionally consisted of metallic cage-like structures. The present vertebral prosthesis 10 may be either used alone to stabilize and fix the spinal column by replacing an excised vertebral body, or alternately may be used in combination with supplemental fixation (not shown), either posterior or anterior, in order to augment the cage fixation. Thus supplemental fixation can include rod and pedicle type screw systems. Typically, if the prosthesis is placed using a posterior approach, the supplemental fixation is also disposed posteriorly. The opposite would be true if an anterior approach is used.

The present vertebral prosthesis (VP) 10 includes opposed first and second end plates 14 and 16 that are interconnected by a generally tubular side wall 12, and which together enclose and define an internal hollow cavity (not shown). The first and second end plates 14 and 16 define outer surfaces 24 and 25 respectively, which form the two outwardly facing surfaces of the VP 10 that are adapted to abut the two adjacent vertebrae 11 and 13. As described further below, the end plates 14,16 are preferably fastened or anchored to the adjacent vertebrae 11,13 using surface features 20 formed on their outer surfaces 24,25. The tubular side wall 12, as will be described further below, has an expanding configuration which allows at least for expansion of the body of the VP 10 such as to fill any sized opening between vertebrae. Particularly, the body of the VP generally may expand along a longitudinal axis 21 of the VP, however it is to be understood that deviations from the axis are of course possible. Regardless, the VP 10 expands such that the end plates 14,16 are generally displaced away from each other. However, the two end plates 14,16 need not remain parallel to each other, and therefore the body can expand to accommodate any slope of the endplates 14,16 necessary for their outer surfaces 24,25 to abut the adjacent vertebrae 11,13, even a slope that is significantly canted from a plane which is perpendicular to the longitudinal axis 21 of the cage.

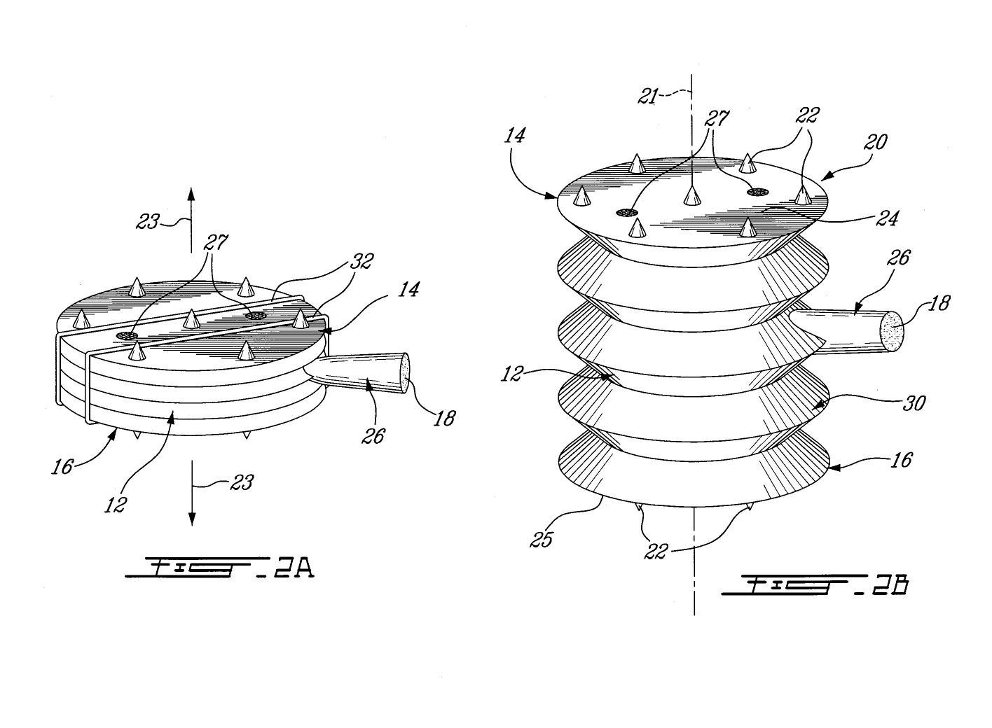

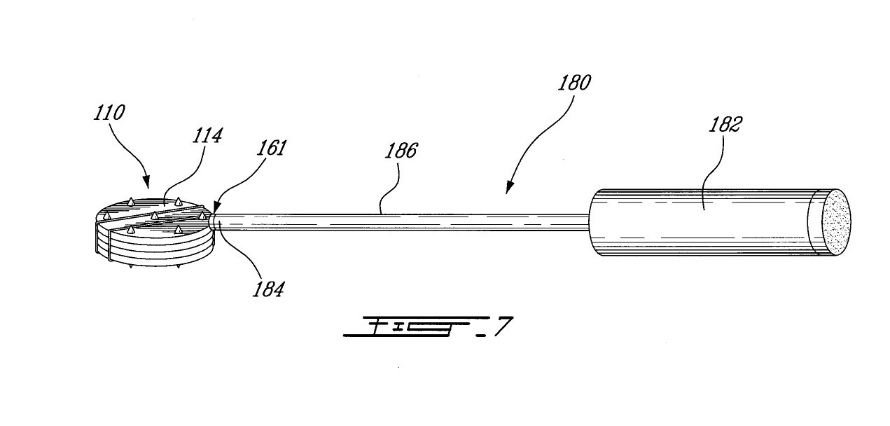

The VP 10 includes a filler inlet port 18 which is disposed in fluid flow communication with the internal cavity within the VP. The filler inlet port 18 may be disposed, for example, in the side wall 12 as shown in FIG. 1, or alternately proximate one of the two end plates 14,16 as shown in the embodiment of FIG. 7, or between an endplate and the side wall. Other positions of the filler inlet port 18 are of course also possible, provided that the inlet port is disposed in fluid flow communication with the internal cavity defined within the VP 10. The filler inlet port 18 is used to inject a hardenable fluid, such as a polymerizing fluid, into the cavity of the device such as to force the expansion of the VP from a collapsed position, as shown in FIG. 2A, to an expanded position thereof, such as shown in FIG. 2B. Preferably, the polymerizing fluid used is a bone cement paste, which hardens once the VP has been forced into the expanded position sufficient to fill the space left by the excised vertebral body or bodies that the VP 10 is replacing. As will be described in further detail below, the combined axial height (i.e. thickness) of the two end plates 14,16 in the direction of the longitudinal axis 21 is relatively small compared to the total axial length (i.e. height) of the VP. This enables the VP 10 to be compressed into much smaller space envelopes than the devices of the prior art, thus enabling the placement of VP 10 via much smaller surgical access openings, and in particularly enabling the placement of the VP 10 via a posterior approach without causing undue damage to the surrounding nerve and tissue structures.

A filler nozzle 26 (see FIG. 2B) may be engaged in communication with the inlet port 18, whether being integrally formed with the side wall 12 or not. The filler nozzle 26 permits the bone cement or other polymerizing fluid to be injected through the inlet port 18 and into the cavity of the prosthesis 10. In FIG. 1, the filler nozzle has been cut off or otherwise detached, which is typically done after the cavity has been filed with the bone cement. Alternately, the filler nozzle 26 is removably engaged to the side wall, such that it can be detached and removed after use without requiring it to be cut off. Alternately still, a filling tube, which removes the need for a separate filler nozzle, can be directly but removably fastened to the VP in communication with the inlet port 18, thereby removing the need for a separate filler nozzle 26 protruding from the VP 10. As will been seen in the embodiment of FIG. 7, this filling tube can also serve as tool (ex: an insertion handle) used by the surgeon to locate the VP 10 in place between the given vertebrae.

The VP 10 thus provides an implant which can be inserted through a relatively small insertion opening, such as through a small posterior surgical access, between pairs of nerve roots, through a costotransversectomy or a wide transpedicular approach, for example. The VP 10 thus has a collapsed position which defines a small size envelope for ease of insertion, but which can subsequently be expanded to fill a much larger space. This is achieved as the VP 10 has a side wall 12 which has an expanding configuration allowing for expansion of the body of the VP. The side wall 12 may have a variety of different configurations, as described further below, however regardless of configuration, the side wall 12 is such that the end plates 14,16 of the VP 10 are displaced away from each other, when the cavity is expanded by the injection thereof of bone cement, such that the VP expands to fill a given opening between vertebral bodies.

The VP 10 includes first and second end “plates” 14 and 16 which are interconnected by the generally tubular side wall 12, such as to define a cavity within the VP. Although the term “plates” is used to define the end surfaces of the body which makes up the VP, it is to be understood that these plates may be integrally formed with the material of the side wall 12, and may also not necessarily be smooth or flat. The end plates 14 and 16 may also be disposed either externally or internally within an outer sheath or casing made up by the material of the side wall 12 which extends over the plates 14,16 at either end. Thus, the plates can constitute a thin walled material, such metal or a polymer (such as a bioresorbable polymer for example), which is either integral with, or separate and fastened to, the material of the side wall 12. The end plates 14, 16 are however preferably, but not absolutely, harder and/or stiffer than the side wall 12, whether the end plates are made of a different material or not.

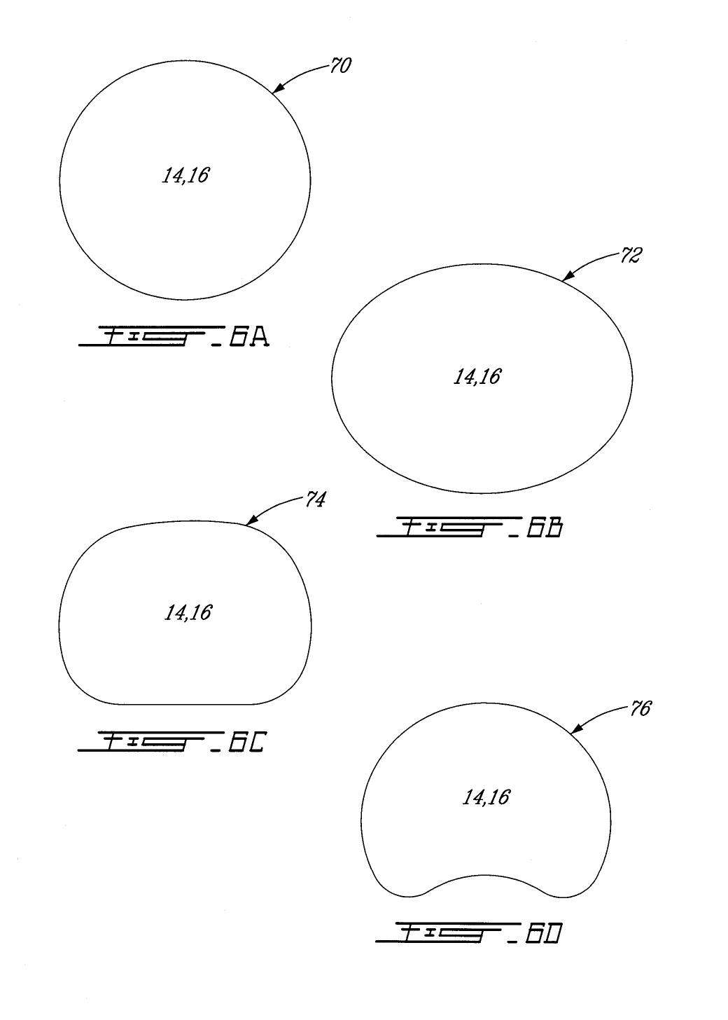

Referring to FIGS. 6A-6D, the end plates 14, 16 may define a shape (when viewed from a top or bottom plan view) which corresponds to that of the central body of the VP made up of the side wall 12. For example, the tubular side wall 12 is generally circular in cross-sectional profile, and therefore the associated end plates 14,16 may be circular end plates 70 as depicted in FIG. 6A. Alternately, however, as shown in FIGS. 6B-6D, the cross-sectional shape or plan profile of the end plates 14,16 can include a number of other possible configurations, such as the oval end plates 72 as shown in FIG. 6B, the D-shaped profile end plates 74 as shown in FIG. 6C, or the kidney (concave-convex) shaped profile end plates 76 as shown in FIG. 6D. The kidney shaped end plate 76 defining an opposed concave-convex shape is advantageous in that is helps to minimize the insertion height/profile, or in other words has a minimized lateral-to-lateral height, which can help to simplify the insertion of the device in place between the vertebrae. In each of these cases, the rest of the VP, i.e. the expanding side wall, may also have a similar cross-sectional profile. In the D-shaped embodiment of FIG. 6C, the flat side of the D-shaped end plate 74 is preferably disposed on the posterior side of the patient, while the curved side of the D-shaped end plate 74 is disposed on the anterior side of the patient. However, it is to be understood that the end plates can be positioned in any manner best suited to accommodate and match the geometry of the patient's vertebrae to which they are to be engaged, as determined and desired by the surgeon. Other cross-sectional shapes of the end plates and the side wall of the VP 10 are also possible, and can be selected based on the desired application and the particularly physiology of the patient.

As best seen in FIGS. 2A to 5, the end plates 14,16 of the VP 10 include, in at least one embodiment, surface features 20 thereon which are adapted to anchor the ends of the VP to the vertebrae 11,13, and/or other biological material, between which the VP is to be located. In one embodiment, these surface features 20 include a plurality of textured protrusions 22 which extend from the outer surface 24 of the end plates 14, 16 such as to permit the end plates to anchor and/or fasten to the bone structures surrounding the VP. These protrusions 22 can include: teeth, pins, barbs, spikes, and any combination thereof. The surface features 20 can also include non-protruding surface feature elements 27, either in addition to or in place of the protrusions 22, which nonetheless help the end plates to be engaged, anchored and/or become fastened to the bone structure of the surrounding vertebrae. These non-protruding elements 27 can include, for example, porous ingrowth surface regions, bioactive bone growth materials, and at least one opening for receiving a bone screw, whereby the end plate is screwed directly in place on the vertebra.

As noted above, the side wall 12 has a configuration which permits expansion of the VP 10 generally in the opposed directions 23, as shown in FIG. 2A, which may in one embodiment be substantially parallel to the longitudinal axis 21 of the VP. Various configurations of side wall 12 are possible to achieve such an expansion, however in the depicted embodiment the side wall 12 has a plurality of accordion type pleats 30 which give the side wall an expanding bellows type folded shape. This folded, tubular side wall 12 thus enables the end plates 14 and 16 to be displaced towards and/or away from each other in a generally longitudinal direction. The accordion pleats 30 of the side wall 12 will prevent the device from unduly expanding in a radial direction and restricts most expansion to the opposed longitudinal directions 23, thus protecting the spinal cord from inadvertent injury when the VP is placed in position between vertebrae and expanded. Further, the flexibility provided by such a wall design permits the two end plates 14 and 16 to be angled, or canted, as required in order to accommodate the specific local topography of the vertebrae against which they are abutted when the VP 10 is expanded in situ. Thus, the end plates 14,16 are free to be disposed, when the VP is expanded in place between the two adjacent vertebrae 11,13, at different angles relative to the longitudinal axis 21 (i.e. the two end plates need not be parallel to each other). The accordion pleat structure of the side wall 12 permits this cant angle mismatch between the two opposed endplates without significant radial displacement of the side walls of the device. In other words, the end plates 14,16 of the VP 10 can automatically (that is, by themselves without requiring outside aid) adjust their angulation to the specific angles of the bone structures to which they are to be attached, as the internal cavity of the VP is filled with the bone cement that forces the two end plates apart from each other and into contact with their adjacent vertebrae. Further, as the VP expands, the bellows structure of the side wall 12 permits the two end plates to be offset from each (in addition to being at different angles) if necessary, i.e. their center points are not axially aligned with each other or with the central longitudinal axis 21. Other expanding wall configurations are possible, in addition to the accordion type design depicted in the figures, such as one having a diamond shaped, braided and/or spiral geometric structure. A Chinese finger trap type orientation of the fibres of the side wall can also be used. Regardless of the particular design, the side wall 12 constitutes a relatively soft pliable shell which is collapsible for insertion of the device and expandable in situ when filled with a suitable hardenable mixture, whether by accordion pleats or by another of the above-mentioned mechanisms.

The side wall 12 may be made of any material that is thin walled and flexible, and suitable for biological applications. These can include metal, plastic or polymer, such as a resorbable polymer for example. The end plates 14,16 may be made of the same material as the side wall 12, or alternately of a different material, such as a more rigid metal, plastic or composite for example.

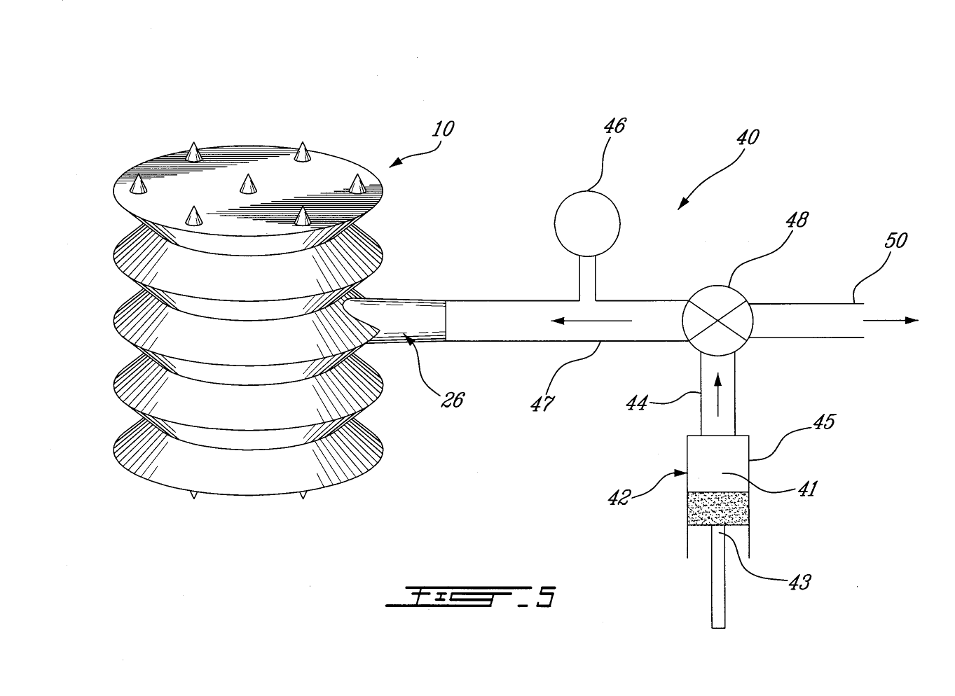

Referring now more specifically to FIGS. 2A and 2B, the fully collapsed position of the VP 10 is shown in FIG. 2A and an expanded position of the VP 10 is shown in FIG. 2B. The VP 10 is placed into position between the vertebrae when in the collapsed position shown in FIG. 2A. Although collapsing straps 32 may be used to help fully compress the VP into as small a package as possible, these are not necessarily required. The natural un-expanded position of the device may be made to be the smallest possible size, and alternately other means may be used to aid in this compression. For example, a vacuum, connected to the filler nozzle 26, may be used to fully collapse the VP. In this fully collapsed position, the VP 10 is sufficiently small to permit its insertion using a posterior approach on most patients. Once installed in position within the space left by the excised vertebral body/bodies which is/are being replaced by the VP 10, a polymerizing fluid injecting system 40 (which will be described further below with reference to FIG. 5) is operatively connected to the filler nozzle 26 such as to inject the polymerizing fluid, such as a cement paste, into the internal cavity defined within the VP 10. As this cavity is filled with the cement paste, the VP is forced to expand in the manner described above such that the two end plates are displaced away from each other in directions 23 and into abutment with the two adjacent vertebrae 11,13 (see FIG. 1). The polymerizing fluid is thus introduced into the device until it has sufficiently expanded to completely fill the space left by the excised vertebral body/bodies which the VP 10 is replacing.

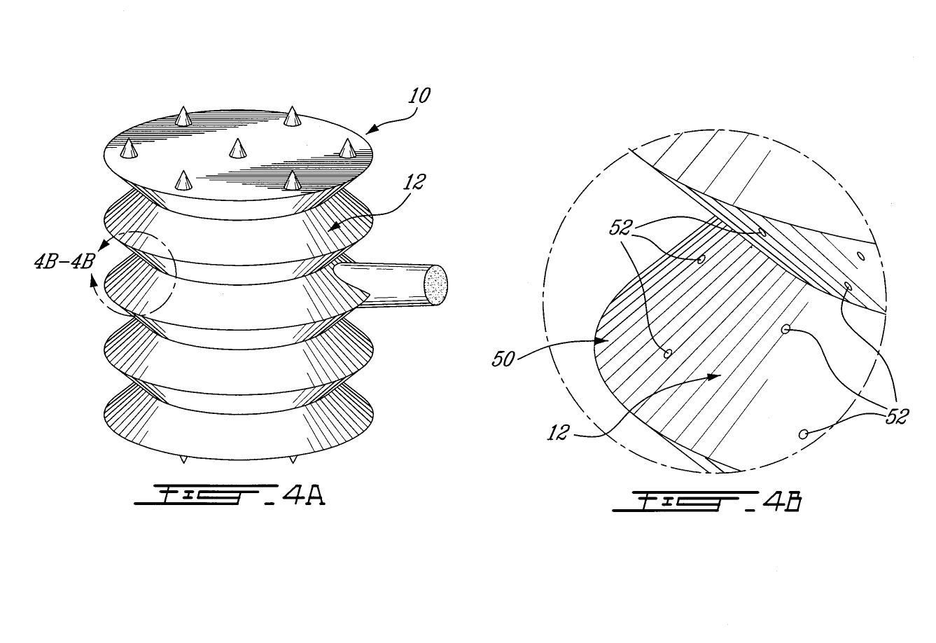

As seen in FIG. 5, the polymerizing fluid injecting system 40 used in one embodiment for injecting the polymerizing fluid, such as bone cement, into the VP 10 includes an injector 42 which may comprise a plunger 43 or syringe type fluid pump or displacement means. The injector 42, when actuated, thus forces the polymerizing fluid 41 from a main reservoir or holding tank 45 through tubing or pipes 44 and 47 and into the VP 10. An overpressure release valve 46 may be provided in the injection line 47, such as to limit and prevent undue over pressurization of the VP 10 with the polymerizing fluid. Thus, significant pressure can be applied such as to expand the VP 10 into an expanded position with the end plates in firmly pressed engagement with the vertebrae, without risk of exceeding a predetermined safe pressure limit Once this preset maximum pressure of the fluid is reached, the pressure relief valve 46 will open in order to relieve the extra pressure in the system. Thus, the endplates 14,16 of the VP 10 can be pumped apart with considerable force using the hydraulic pressure produced by the fluid injector pump 42 to cause expansion of the device 10. Pumping with excess force is limited by the pressure release valve 46, such that over pressurization of the device, and thus possible wall rupture or bone and/or tissue damage, is unlikely. The fluid injecting system 40 may also include, in an alternate embodiment, an additional three-way valve 48 which allows transition from air evacuation (i.e. vacuum generation) to polymerizing fluid injection. Therefore, although the three-way valve 48 is depicted in FIG. 5, the system 40 need not necessary include this three-way valve 48 if the VP 10 includes an alternate air evacuation system as it does in the embodiment described below with reference to FIGS. 4A and 4B. However, if the three-way valve 48 is included, an upstream section of conduit 50 may be connected to a vacuum source, such as to permit evacuation of the air from within the cavity defined within the VP 10, with the valve 48 allowing the outward flow of air from within the cavity. In this embodiment, the walls 12 of the VP 10 are made of a substantially air-tight material which will not permit air to evacuate therethrough. Thus, when a vacuum line or source is connected to the conduit 50, this draws the air out from the cavity of the VP 10, typically prior to the injection of the cement into the VP using the injector 42. The three-way valve 48 permits both the air evacuation through the exit conduit 50 and the flow of cement through the passages 44 and 47 before being injected into the VP 10. The vacuum can be used to first completely evacuate all air from within the VP 10, before the cement is injected therein using the injector pump 42 of the injection system 40. Thus, the three-way valve 48 is first turned to allow vacuum creation within the VP 10. The vacuum created is sufficient to completely collapse the device 10 into the fully collapsed position as shown in FIG. 2A, such that it can be readily placed into the desired position. The valve 48 is then activated to maintain this vacuum within the device. Once the device is in position, the VP is slowly pumped full of the polymerizing cement until it has reached the maximum possible expansion given the available anatomical space (as shown in FIG. 1, for example). The cavity under vacuum will suck cement into itself during this process, thus eliminating air voids therewithin as it is filled with the polymerizing cement.

However, in one embodiment, the VP 10 includes an integrated air evacuation system, thus making this additional three-way valve 48 and vacuum source unnecessary, at least as a primary air evacuation means. As seen in the embodiment of FIGS. 4A and 4B, the VP 10 has an integrated micropore air evacuation system 50, comprising a plurality of microscopic, or at least very small, air evacuation holes 52 defined through the side wall 12 of the VP 10. These air evacuation holes 52 permit the evacuation of air from the internal cavity within the VP 10, such as to prevent air entrainment. The holes 52 are however sufficiently small in size to prevent the relatively viscous polymerizing fluid (ex: cement) from escaping from the internal cavity through the walls of the VP 10, as this polymerizing fluid is significantly more viscous than air. However, it is to be understood that the device 10 can include such an integrated micropore air evacuation system 50, while still employing a fluid injecting system 40 that includes the three-way valve 48 described above.

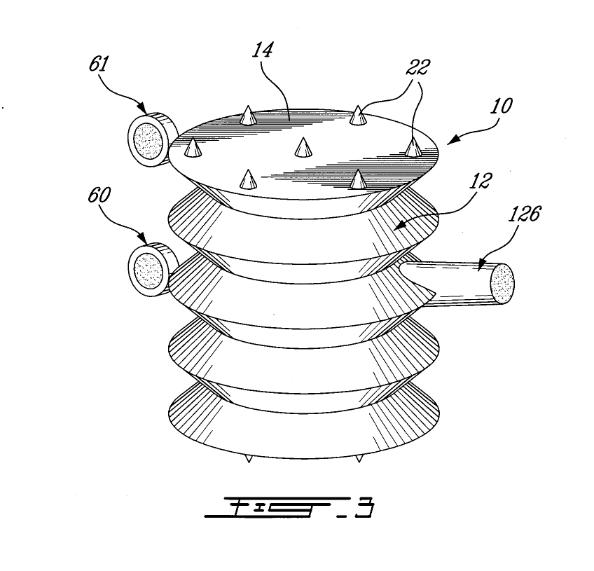

Referring now to FIG. 3, the VP 10 is shown with an alternate filling nozzle 126 fixed thereon. The filling nozzle 126 is as the nozzle 26 described above, however the filling nozzle 126 is of a so-called “fold flat” design, and is able to collapse such as to permit the entire VP device to collapse more completely when in the fully collapsed position as shown in FIG. 2A. The filling nozzle 126 may thus be formed of an elastic or flexible material which is able to be compressed into a relatively flatter shape, while still being able to re-open into an injecting nozzle sufficient to feed the polymerizing polymerizing fluid into the VP 10 upon expansion thereof.

As seen in FIG. 3, the VP 10 may also be provided with at least one protruding tab which is used to help position and locate the device in place within the patient. For example, a first tab 60 for pedicle screw engagement and/or capture may be provided. Although the tab 60 is shown fixed to the side wall 12 of the device, it can be located as necessary in order to effectively engage a mating rod or screw used to position and retain the device in place. Thus, a pedicle screw and/or rod of a pedicle screw/rod system can be mated with the protruding tab 60, in order to fasten the VP 10 either directly to a vertebra or to another element (such as a rod) of the pedicle screw/rod/hook system, in cases where the surgeon desires additional fixation of the VP 10 in addition to the engagement between the surface features 20 of the end plates 14,16 and the abutting vertebrae. This provides additional secure attachment used to fasten the entire VP device 10 in place, and helps to prevent any possible unwanted migration of the VP 10 out of its desired installation position.

Further, a second tab 61 may also be provided, and in the depicted embodiment this second tab 61 is engaged to the endplate 14. The second tab 61 provides an additional attachment point for a cage holder, rod or screw used to anchor the device 10 in place. For example, the second tab 61 may provide a secondary posterior fixation point for fastening the VP 10 to the surrounding bone structure. Although the second tab 61 is schematically depicted in FIG. 3 as being fixed to a specific point on the upper end plate 14, the tab 61 may be located at any point about the perimeter of the end plate, and may be pivoted in place such as to be orientated at an appropriate angle to permit mating engagement with an associated fastener, rod, or the like. For example, the tab 61 may be mounted to a ring (not shown) which is capable of being rotated about the periphery of the end plate, in addition to be pivotably mounted to the ring such as to permit rotation about its own transverse axis extending across the diameter of the circular tab 61. The second tab 61 may also form part of an injection port to the cavity within the VP 10, such as in the embodiment of FIG. 7 described below.

In use, the VP 10 collapses into a very small size envelope, such as to make its insertion into place between the nerve roots of two adjacent vertebrae possible without causing damage, even upon a posterior placement. Although the distance between adjacent nerve roots varies along the spine, this distance is generally between about 1 cm and about 2 cm. Accordingly, when the VP 10 is disposed in its fully collapsed position, it has a total collapsed height of less that about 1-2 cm.

As the end plates 14,16 are very thin relative to the total potential height of the entire device 10, the fully collapsed position (FIG. 2A) can be much smaller than most existing cage designs of the prior art, particularly relative to their expansion potential. For example, in one embodiment, a combined axial height of the end plates 14,16 is at most 20 percent of the total height of the VP 10 when it is disposed in the fully collapsed position, as shown in FIG. 2A for example. Thus, in this embodiment, the tubular side wall 12 thereby has an axial height of at least 80 percent of the total height of the VP, in the fully collapsed position. In another more specific embodiment, the combined axial height of the end plates makes up at most 10 percent of the total height of the device, when the VP 10 is disposed in the fully collapsed position. Thus, in this embodiment, the tubular side wall 12 thereby has an axial height of at least 90 percent of the total height of the VP when disposed in its most collapsed position. It is to be understood that the term “total height” as used herein is intended to mean the longitudinal axial distance between the outer surfaces of the first and second end plates 14,16 of the VP 10. Thus, for a similarly sized fully expanded height, the fully collapsed height of the VP 10 is much small than those of the prior art.

Thus, one feature of the VP 10 is that it can be greatly collapsed, permitting significantly higher expansion ratios (i.e. the total expanded height divided by the collapsed height), such as expansion ratios ranging from about 200% to over 500%. In one particular embodiment of the VP 10, this expansion ratio is at least 200%. In another embodiment, the expansion ratio is greater than 250%. In another embodiment, the expansion ratio is greater than 300%. In yet another specific embodiment, the expansion ratio is between 400 and 500%. This is at least partly possible due to the relatively thin end plates. In one embodiment, the combined axial height of the first and second end plates is less than 5% of the total height of the entire VP 10 when it is disposed in the expanded position, as shown in FIG. 2B. This compares to prior art designs, in which at least 65% of the overall height of the entire cage is taken up by the thick endplates. For such prior art designs, expansion ratios are also much smaller, such as of the order of about 140%.

In one possible example, these end plates 14,16 are about 3 mm thick each and the height of the collapsed bellows side wall 12 is about 54 mm, for a total collapsed height of 60 mm for the VP 10. Given that the bellows-like side wall 12 of the device can expand about 300% (i.e. 54 mm×3=162 mm), then the VP 10 would be able to expand to a total height of about 168 mm (i.e. 3 mm+162 mm+3 mm) This corresponds to an expansion ratio of about 280%. Thus, for a given total collapsed height, the present VP 10 is capable of expanding to fill a much bigger defect gap than is possible with any device of the prior art. As such, the VP 10 is capable of being expanded to fill a gap left by 1, 2 or 3 excised vertebrae, for example.

If viewed in an alternate manner, given a fairly typical 70 mm vertebral defect height which can exist after a thoracolumbar or lumbar vertebrectomy for example, the VP 10 of the present invention could be inserted therein having a 21.3 mm minimum (i.e. collapsed) height, which enables its insertion between pairs of nerve roots from a posterior approach, prior to being sufficiently expanded to adequately fill the 70 mm defect opening. In another embodiment of the present invention, the VP 10 has an overall expansion ratio of 400-500%, which results in an minimum entry height of the collapsed device about 14 mm possible for a 70 mm space. This is clearly a large improvement over the devices of the prior art, wherein the expandable cages would typically have a minimum (collapsed) height of 60 mm, which is far too large to be inserted from the posterior approach.

Further, the VP 10 lacks any dynamic end plate design, and therefore does not permit relative movement between the end plate fixed to the vertebrae and the rest of the VP. Thus, in the VP 10, the end plates 14,16 become rigid, and therefore fixed in position relative to the side wall 12, once the polymerization fluid (cement) has been injected into the body and hardens. Therefore, although the two end plates can be displaced and angled relative to each other as needed prior to the polymerization fluid hardening, once this cement has hardened the VP 10 forms a single, fixed structure. Thus, contrary to certain prior art designs which include a viscoelastic coupling within large end plates of the vertebrae cages, no relative mobility between the end plates 14,16 and the side wall 12 of the present VP 10 is possible once the cement has hardened and the VP 10 becomes a single rigid body interconnecting the adjacent vertebrae, effectively fusing them together into a single, linked, rigid body.

Referring now to FIG. 7, a collapsed VP 110 in accordance with another embodiment is depicted in engagement with an associated insertion handle 180. The VP 110 is as per the VP 10 described above, however includes a single filling and mounting point 161. The VP 100 therefore may not include the centrally mounted filling nozzle 26 in the side wall of the expandable device, although it remains possible to nevertheless include such a secondary filling nozzle 26 in the event that a backup filling point is required. An insertion handle 180 is used to manipulate the VP 100 in order to permit it to be inserted in place between the vertebrae of the patient. The insertion handle 180 includes a grip portion 182 on an outer end thereof, which the surgeon holds for manipulation of the handle 180 and therefore of the VP 110 removably fastened to an inner end 184 thereof. The inner end 184 is removably fastened with the mounting point 161 on one of the two end plates (such as the upper end plate 114 shown) of the VP 110. This removable engagement may be by threaded engagement or by another suitable engagement means, such as a sealing quick connect coupling for example.

In the present embodiment, the mounting point 161 on the endplate 114 of the VP 110 also serves as the inlet filling port to the internal cavity of the VP 110. Accordingly, the body 186 of the insertion handle 180 is hollow and defines therethrough a conduit through which the hardenable polymerizing fluid (ex: bone cement) is fed in order to be injected into the cavity of the VP 110. The inner end 184 of the handle 180 is therefore mated with the mounting point 161 on the device in fluid flow communication, with the mounting point 161 providing fluid flow communication with the internal cavity of the device. As such, the insertion handle 180 acts as both a tool used to manipulate and position the VP 110 in a desired position, and as a polymerizing fluid injection conduit via which the polymerizing fluid is fed from a pressurized source thereof into the cavity within the expandable VP 110. In a particular embodiment, the grip portion 182 includes a control device for regulating the flow of the polymerizing fluid through the conduit within the insertion handle 180 and therefore the flow into the VP 110. For example, the handle's grip 182 itself may form a pump used by the surgeon to pump the polymerizing fluid through the conduit within the handle, or alternately may be rotated or otherwise displaced in order to actuate a fluid control valve integrated thereon and which acts to vary the flow of polymerizing fluid into the VP 110. Once the polymerizing fluid is fed into the cavity of the VP 110, thereby forcing the VP 110 to expand to fit within the vertebral cavity required, the flow of fluid is stopped and the inner end 184 is then detached from the VP 110.

The embodiments of the invention described above are intended to be exemplary. Those skilled in the art will therefore appreciate that the forgoing description is illustrative only, and that various alternatives and modifications can be devised without departing from the spirit of the present invention. Accordingly, the present is intended to embrace all such alternatives, modifications and variances which fall within the scope of the appended claims.

1. A vertebral prosthesis comprising opposed first and second ends interconnected by a tubular side wall, the first and second ends and the tubular side wall enclose a cavity therewithin, at least one inlet port in fluid flow communication with said cavity permits injection of a hardenable fluid into said cavity, the first and second ends including outer surfaces thereon which are respectively adapted to abut adjacent vertebral bodies for engagement therewith, the tubular side wall having an expanding bellows configuration which allows expansion of the vertebral prosthesis in an axial direction such that the first and second ends are displaced away from each other when the cavity is filled with the hardenable fluid, the vertebral prosthesis being thereby axially expandable from a collapsed position to an expanded position in order to fill a space between said adjacent vertebral bodies, the vertebral prosthesis having an expansion ratio defined by a total axial height of the vertebral prosthesis in the expanded position divided by a total axial height of the vertebral prosthesis in the collapsed position, the expansion ratio being greater that 200 percent.

2. The vertebral prosthesis as defined in claim 1, wherein the expansion ratio is greater than 250 percent.

3. The vertebral prosthesis as defined in claim 2, wherein the expansion ratio is greater than 300 percent.

4. The vertebral prosthesis as defined in claim 3, wherein the expansion ratio is between 400 and 500 percent.

5. The vertebral prosthesis as defined in claim 1, wherein said outer surfaces of the first and second ends include surface features thereon for fastening engagement with said adjacent vertebral bodies.

6. The vertebral prosthesis as defined in claim 5, wherein said surface features include at least one of teeth, pins, barbs, spikes, porous ingrowth surface regions and bioactive bone growth materials.

7. The vertebral prosthesis as defined in claim 1, wherein the tubular side wall is composed of at least one of a polymeric material, a metallic material and a bioresorbable material.

8. The vertebral prosthesis as defined in claim 7, wherein the first and second ends are composed of the same material as the tubular side wall.

9. The vertebral prosthesis as defined in claim 1, further comprising an air evacuation system for evacuating air from out of the cavity.

10. The vertebral prosthesis as defined in claim 9, wherein the air evacuation system includes microscopic holes defined in the tubular side wall to permit air evacuation therethrough while preventing the more viscous hardenable fluid to flow therethrough.

11. The vertebral prosthesis as defined in claim 1, wherein an insertion handle is removably engageable to the vertebral prosthesis, the insertion handle being operable to manipulate the vertebral prosthesis into position and having a conduit defined therethrough, the conduit being in fluid flow communication with said inlet port of the vertebral prosthesis such that the hardenable fluid can be fed through the insertion handle for injection into said cavity.

12. The vertebral prosthesis as defined in claim 1, wherein at least one protruding tab is engaged to the vertebral prosthesis, said tab providing a fixation point for fastening the vertebral prosthesis to a surrounding structural component in order to locate the vertebral prosthesis in place.

13. A vertebral prosthesis for replacement of at least one vertebral body excised from between two other vertebral bodies, the vertebral prosthesis comprising:
opposed end plates including outer surfaces thereon which face in opposite directions and are respectively adapted to abut said two other vertebral bodies for fastening engagement therewith;
a tubular side wall interconnecting the end plates to define an enclosed cavity therewithin, the tubular side wall having an expanding configuration allowing expansion of the vertebral prosthesis in a axial direction such that the end plates are displaced away from each other, when said cavity is filled with a hardenable fluid, such that the vertebral prosthesis expands from a collapsed position to an expanded position thereof in order to fill a space left by the at least one excised vertebral body; and
wherein the vertebral prosthesis has an expansion ratio defined by a total axial height of the vertebral prosthesis in the expanded position divided by a total axial height of the vertebral prosthesis in the collapsed position, the expansion ratio being greater that 200 percent.

14. The vertebral prosthesis as defined in claim 13, wherein a combined axial height of said end plates in said axial direction is at most 20 percent of the total axial height of the vertebral prosthesis in said collapsed position, the tubular side wall having an axial height of at least 80 percent of the total axial height of the vertebral prosthesis in said collapsed position.

15. The vertebral prosthesis as defined in claim 13, wherein a filler inlet port is disposed in fluid flow communication with said cavity, the filler inlet port being adapted to direct the hardenable fluid into the cavity to force the expansion of the vertebral prosthesis into said expanded position.

16. The vertebral prosthesis as defined in claim 13, wherein a combined axial height of said end plates is less than 10 percent of the total axial height of the vertebral prosthesis when disposed in said collapsed position, said tubular side wall having an axial height of at least 90 percent of the total height of the vertebral prosthesis in said collapsed position.

17. The vertebral prosthesis as defined in claim 13, wherein a combined axial height of said end plates is less than 5 percent of the total height of the vertebral prosthesis when disposed in said expanded position.

18. The vertebral prosthesis as defined in claim 13, wherein a combined axial height of said end plates is about 6 mm.

19. The vertebral prosthesis as defined in claim 13, wherein the expansion ratio is greater than 250 percent.

20. The vertebral prosthesis as defined in claim 19, wherein the expansion ratio is greater than 300 percent.

21. The vertebral prosthesis as defined in claim 20, wherein the expansion ratio is between about 400 and 500 percent.

22. The vertebral prosthesis as defined in claim 13, wherein the tubular side wall comprises expanding bellows.

23. The vertebral prosthesis as defined in claim 13, wherein said outer surfaces of the end plates include surface features thereon for fastening engagement with said two other vertebral bodies.

24. The vertebral prosthesis as defined in claim 23, wherein said surface features include at least one of teeth, pins, barbs, spikes, porous ingrowth surface regions and bioactive bone growth materials.

25. The vertebral prosthesis as defined in claim 13, wherein the tubular side wall comprises at least one of a polymeric material, a metallic material and a bioresorbable material.

26. The vertebral prosthesis as defined in claim 13, wherein the ends plates are composed of the same material as the tubular side wall.

27. The vertebral prosthesis as defined in claim 13, further comprising an air evacuation system for evacuating air from out of the enclosed cavity.

28. The vertebral prosthesis as defined in claim 27, wherein the air evacuation system includes microscopic holes defined in the tubular side wall to permit air evacuation therethrough while preventing more viscous polymerizing fluid to flow therethrough.

29. The vertebral prosthesis as defined in claim 13, wherein an insertion handle is removably engageable to an inlet port in fluid flow communication with the cavity, the insertion handle being operable to manipulate the vertebral prosthesis and having a conduit defined therethrough, the conduit being in fluid flow communication with said inlet port such that the hardenable fluid can be fed through the insertion handle for injection into said cavity.

30. The vertebral prosthesis as defined in claim 13, wherein at least one protruding tab is engaged to the vertebral prosthesis, said tab providing a fixation point for fastening the vertebral prosthesis to a surrounding structural component in order to locate the vertebral prosthesis in place.
